(19) United States
(12) Patent Application Publication
(10) Pub. No.: US 2011/0295289 A1
(43) Pub. Date: Dec. 1, 2011

(54) MICROSURGERY CLAMP, IN PARTICULAR MICROINCISION CAPSULORHEXIS CLAMP

(76) Inventor: Jean-Marie André, Marseille (FR)

(21) Appl. No.: 13/142,878

(22) PCT Filed: Dec. 17, 2009
(86) PCT No.: PCT/FR2009/001439

§ 371 Date: Aug. 13, 2011

(30) Foreign Application Priority Data
Jan. 27, 2009 (FR) 09/00349

(51) Int. Cl. A61B 17/08

(52) U.S. Cl. 606/157

(57) ABSTRACT

Microsurgery clamp, particularly a microincision capsulorhexis clamp, having a proximal handling portion or handle including two branches or arms interconnected at one of the ends thereof, so as to be suitable for being approached or separated elastically, and a distal clamping portion having two narrow and thin clamping blades connected to the distal ends of the handling branches and oriented laterally relative thereto. The distal end of each of the clamping blades being curved in a direction opposite to that of the handle, so as to form a clamping tip. The clamping blades are oriented in a direction or plane corresponding to the approaching or separation directions of the handling branches. The clamping blades are arranged above each other, relative to a vertical position of said handle, so that the approaching or separation movement of said handling branches results in the approaching or separation movement of said clamping tips, respectively.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to the field of surgery and finds a particularly advantageous application in that of cataract procedures. More specifically, it relates to a precision surgical clamp which may particularly be a capsulorhexis clamp.

2. Description of Related Art Including Information Disclosed Under 37 CFR 1.97 and 37 CFR 1.98.

Cataract procedures comprise a surgical step commonly referred to as “capsulorhexis”, consisting of making a continuous circular opening in the anterior lens capsule. During this step, a special clamp, referred to as a capsulorhexis clamp, is inserted via a corneal or sclerocorneal incision to the anterior lens capsule, where it grips a piece to produce a continuous circular opening therein. After the removal thereof, the torn portion leaves room for the desired opening.

More specifically, according to one known embodiment (for example U.S. Pat. No. 5,167,618), a capsulorhexis clamp comprises two symmetrical branches interconnected at one of the ends thereof and the opposite ends whereof consist of clamping tips curved in a direction perpendicular to the approach plane of said branches, said extremely fine clamping tips thus being suitable for being approached elastically with respect to each other and forming the active distal portion of said clamp.

A capsulorhexis clamp of this type is represented in FIG. 1 of the appended drawings.

It is conceivable that it is desirable for the corneal or sclerocorneal incision to be as small as possible.

At the present time, rhexis clamps of the type mentioned above can operate via a corneal incision at least 3 mm in length.

However, a reduction in the size of this incision is desirable, particularly to avoid sutures, promote healing and obtain superior postoperative results.

However, while a conventional rhexis clamp can operate via a 3 mm corneal incision, the operation thereof becomes difficult or impossible via an opening of lesser length. Indeed, in this case, the clamping tips, after being inserted in the approached position into said incision (FIG. 3), would not have enough room to be able to separate (FIG. 4), to be able to seize a piece of the anterior capsule and release it after producing the opening in said capsule.

Developments in surgery techniques are generally tending towards a reduction in the size of the incisions to access the organs undergoing the procedure and, in the case of cataract procedures, a reduction in the corneal or sclerocorneal incision.

### BRIEF SUMMARY OF THE INVENTION

The aim of the invention is particularly that of providing eye surgeons with a rhexis clamp capable of carrying out the function thereof via a 1.5 mm corneal opening.

According to the invention, this aim is achieved by means of a microsurgery clamp, in particular a microincision capsulorhexis clamp, comprising a proximal handling portion or handle including two branches or arms interconnected at one of the ends thereof, so as to be suitable for being approached or separated elastically, and a distal clamping portion having two narrow and thin clamping blades connected to the distal ends of the handling branches and oriented laterally relative thereto, the distal end of each of the clamping blades being curved in a direction opposite to that of the handle so as to form a clamping tip, said clamp being characterised in that the clamping blades are asymmetrical and oriented in a plane corresponding to the approaching or separation directions of the handling branches, said clamping blades being arranged above each other, so that the approaching or separation movement of said handling branches results in the approaching or separation movement of said clamping tips forming the jaws of the clamp, respectively.

According to one advantageous embodiment, the clamping blades are designed to be able to slide over each other during handling branch approaching and separation movements.

According to a further advantageous embodiment, at least one of the clamping blades is made of an elastically flexible material. Preferably, both clamping blades are made of an elastically flexible material.

According to a further advantageous feature, the clamping blades have a curved shape.

According to a further characteristic feature, the radius of curvature of the upper clamping blade is smaller than the radius of curvature of the lower clamping blade.

According to a further characteristic feature, the upper clamping blade is shorter than the lower clamping blade.

According to a further characteristic feature, the clamping tip or jaw of the lower clamping blade has, in the upper portion thereof, a greater width than that of the distal portion of the upper clamping blade, so as to protrude on either side thereof.

According to one preferred embodiment, the clamping tips have a triangular shape and the lower portions thereof are suitable for stacking, in the approaching position.

It is understood that, in the capsulorhexis procedure step, the microsurgery clamp according to the invention can fulfil the function thereof via a very small corneal incision, less than 2 mm, for example via an incision of only 1.5 mm.

Indeed, the width of the invasive portion of the clamp, consisting of the stacked curved portions of the clamping blades, does not vary during the handling of the clamp, whether said clamp is open or closed.

Furthermore, despite the high precision thereof, said microsurgery clamp can be manufactured industrially in the form of a single-use, disposable item.

### BRIEF DESCRIPTION OF THE DRAWINGS

The above aims, features and advantages, along with others, will emerge more clearly from the description hereinafter and the appended figures wherein:

FIG. 1 is a perspective view of a conventional rhexis clamp.

FIG. 2 is a top view of said clamp.

FIGS. 3 and 4 are perspective and larger scale detailed views, illustrating the operation of a conventional rhexis clamp.

FIG. 5 is a perspective view of a rhexis clamp according to the invention, represented in an open position.

FIG. 6 is a top view of said clamp.

FIG. 7 is a side view thereof.

FIG. 8 is a side view showing the approach of the handling branches and the clamping tips of the clamp, in a position for gripping the anterior lens capsule.

FIG. 9 is a larger scale cross-sectional view along the line 9-9 in FIG. 8.

FIG. 10 is a side view representing the clamp in the fully closed position.

FIG. 11 is a larger scale cross-sectional view, along the line 11-11 in FIG. 10.

FIG. 12 is a larger scale perspective view of the distal ends of the clamping blades and the clamping tips thereof.

FIG. 13A is a larger scale, side detailed view, showing the distal ends of the clamping branches of the clamp, represented in the initial approach position shown in FIG. 8.

FIG. 13B is a view similar to FIG. 13A showing the clamp in the fully closed position represented in FIG. 10.

FIGS. 14A and 14B are top views of FIGS. 13A and 13B, respectively.

FIGS. 15 and 16 are perspective views illustrating the intra-ocular operation of a rhexis clamp according to the invention.

Reference is made to said figures to describe an advantageous example of an embodiment, which is in no way limitative, of the microsurgery clamp according to the invention.

### DETAILED DESCRIPTION OF THE INVENTION

Furthermore, a particularly advantageous application of the invention to the embodiment of a rhexis clamp is described hereinafter, but it should be noted that the invention can obviously be implemented for the production of microsurgery clamps more specifically suitable for performing other surgical procedures liable to be carried out via a very small incision.

It is noted that, for easier understanding of the invention, in the description and in the claims, terms such as “upper”, “lower”, “above”, “below”, “high”, “low” are used, with reference to the vertical or approximately vertical position of the microsurgery clamp handle in a rhexis procedure; these terms are thus in no way limitative.

The rhexis clamp according to the invention is, preferably, of the type essentially comprising: a proximal handling portion 1 or handle including two branches or arms 1a, 1b, interconnected at one of the ends thereof, so as to be suitable for being approached or separated elastically, and a distal clamping portion 2 having two narrow and thin clamping blades 2a, 2b connected to the distal ends of the handling branches 1a, 1b and oriented laterally relative thereto, the distal end of each of said clamping blades 2a, 2b being curved in a direction opposite to that of the handle 1, so as to form a clamping tip 3a, 3b.

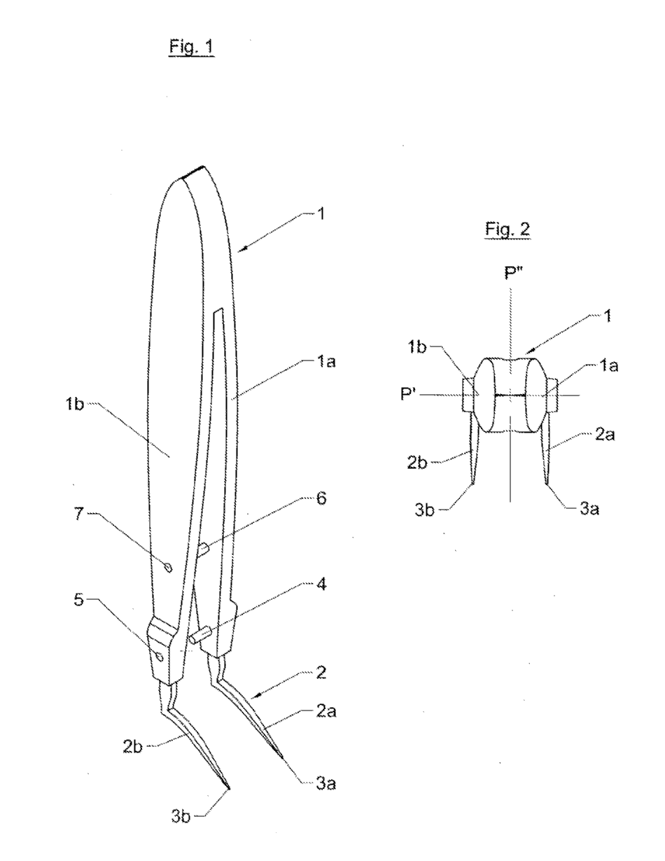

As shown in FIG. 2, the clamping blades 2a, 2b, of such a conventional eye microsurgery clamp are oriented laterally in a direction or plane P″ perpendicular to the direction or plane P′ wherein the branches 1a, 1b of said clamp are approached and separated. Moreover, such a clamp may be advantageously provided with centering and pre-centering means comprising lugs or pins 4, 6 rigidly connected to one of the branches and oriented in the direction of the other branch, and of orifices 5, 7 provided in the other branch, opposite said lugs or pins.

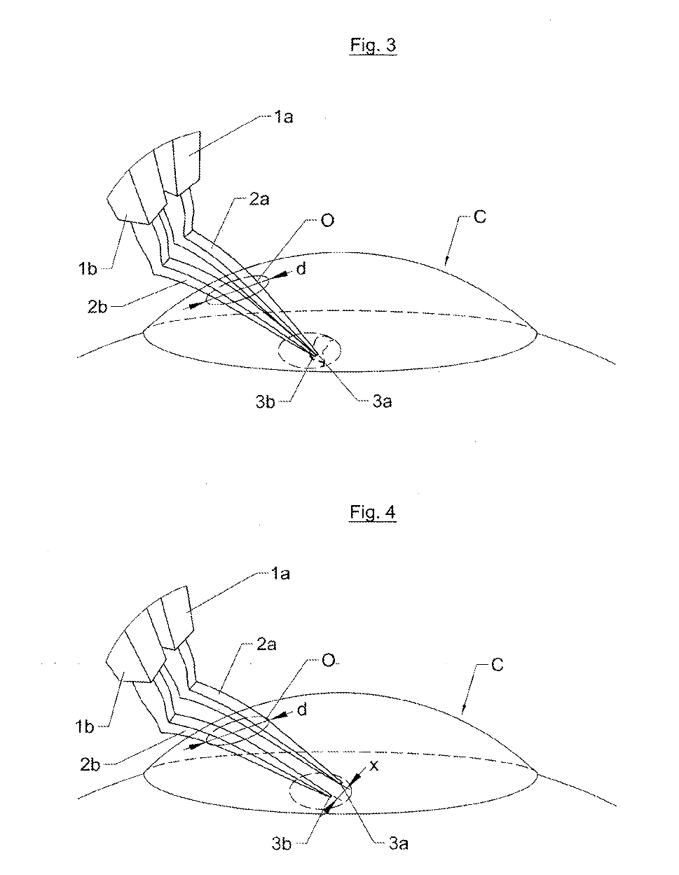

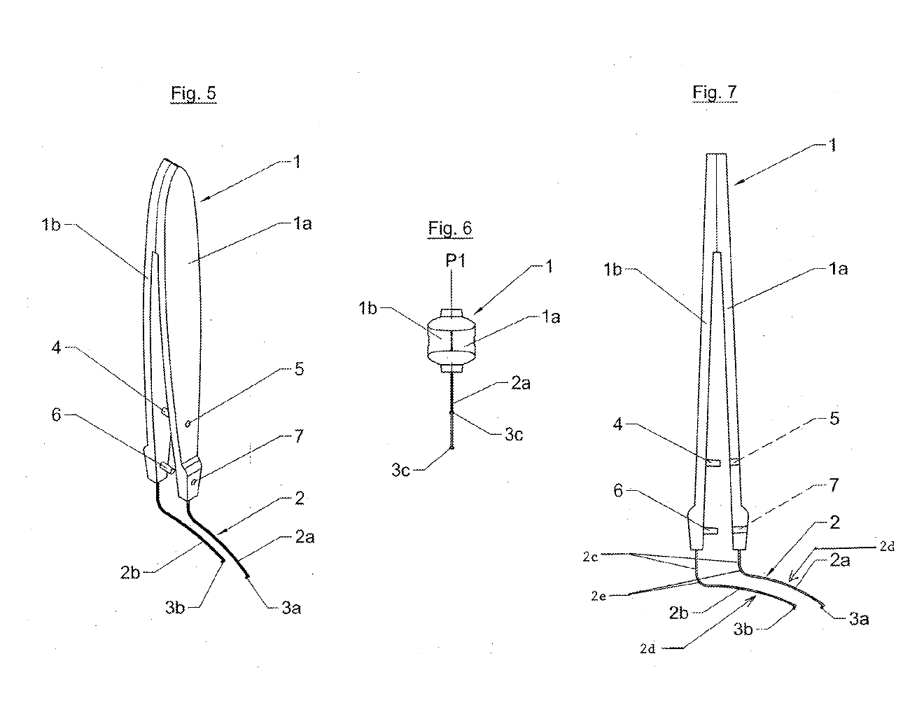

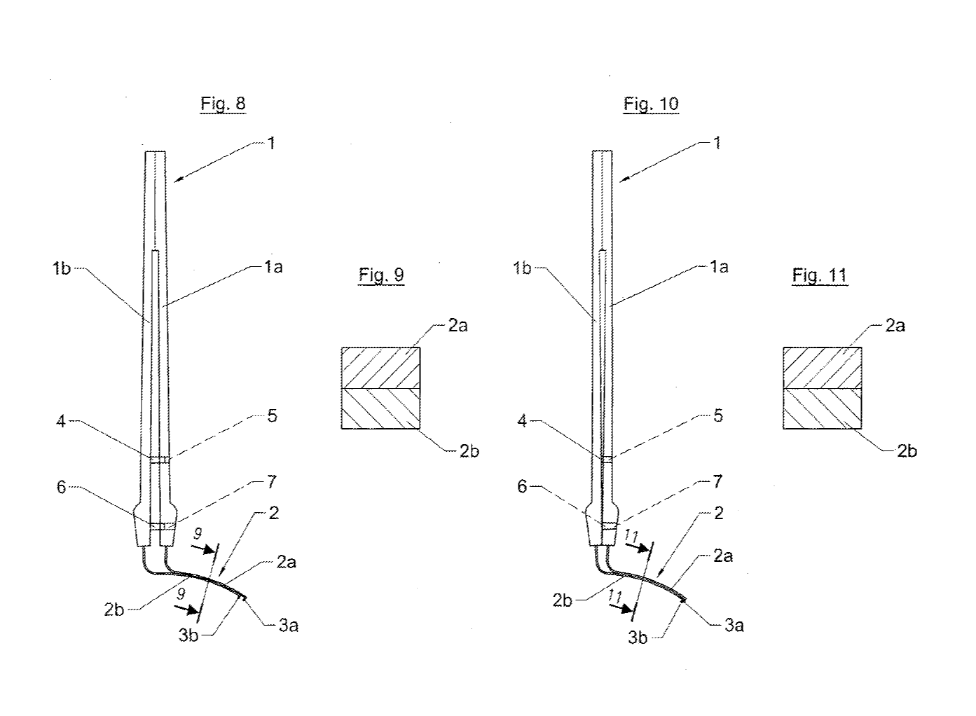

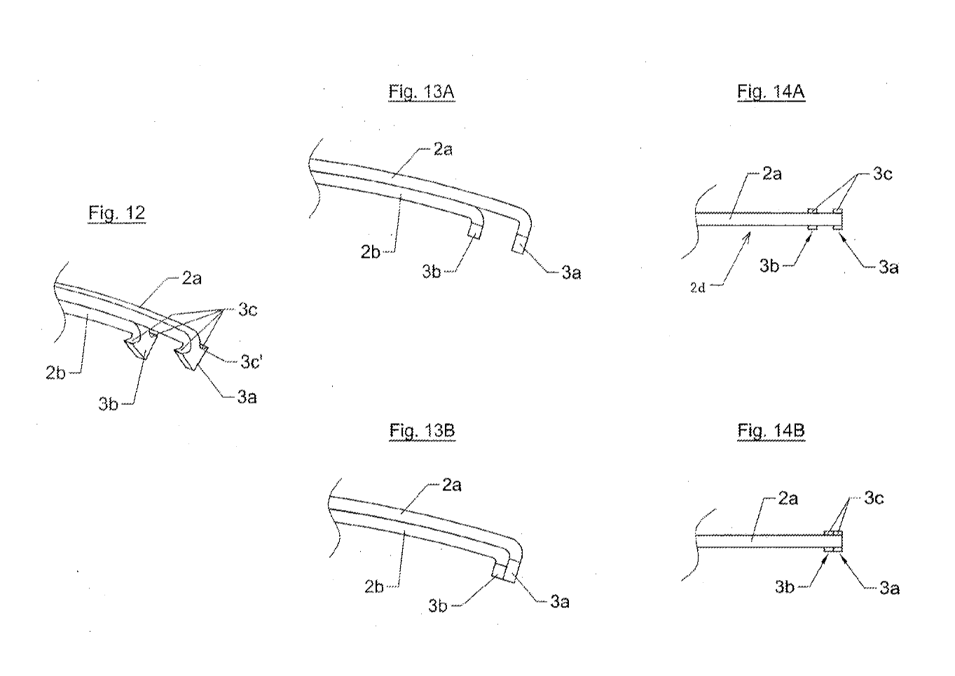

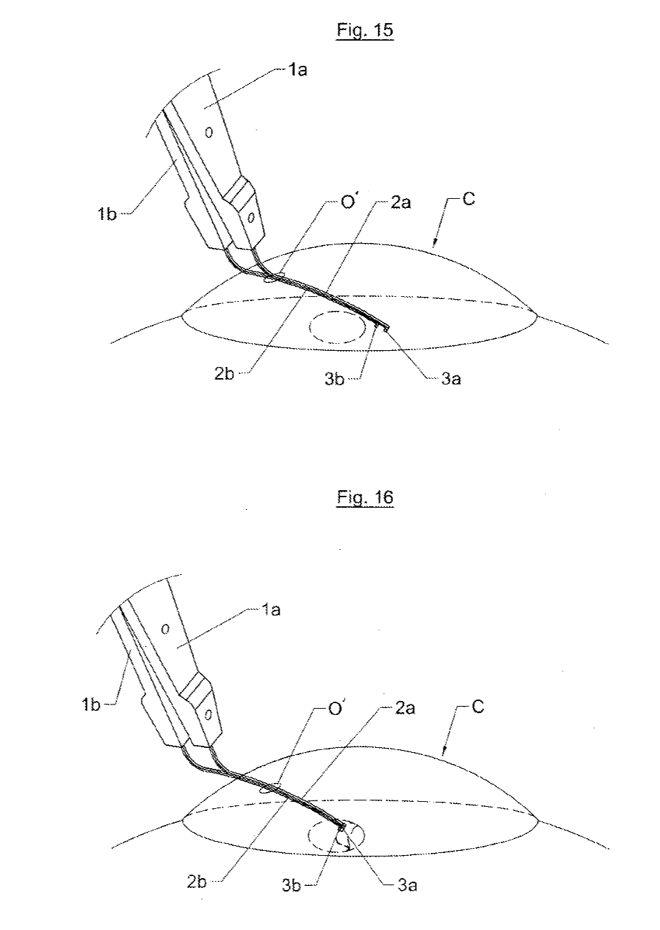

A conventional rhexis clamp of this type is represented in FIG. 1 and the operating mode thereof is illustrated in FIGS. 3 and 4.

Initially, the rhexis clamp is idle and is not subjected to any pressure force; the clamping blades 2a, 2b are separated from each other. By applying simultaneous pressure on the two branches 1a, 1b of the clamp, the ends of the clamping blades 2a, 2b are brought into contact with each other. Said clamping blades 2a, 2b are then inserted in this position, into the eyeball, via the corneal incision O (FIG. 3) previously produced in the cornea C, the size d whereof necessarily being greater than or equal to 3 mm. Indeed, the clamping ends or portions 3a, 3b of the clamping blades 2a, 2b should then be separated by a distance X (FIG. 4) to be able to grip the piece of anterior capsule (previously created, during a previous procedure phase). It is understood that the length d of the incision O should necessarily, to enable the separation of the clamping blades 2a, 2b, inside the eye, be greater than the length of the incision required merely to insert said clamping blades in the approached position, into the eye. To enable this separation X, the incision or opening O must be at least 3 mm in size, as explained above, this size corresponding to the distance d between the outer faces of the clamping blades 2a, 2b, in the separation position thereof (FIG. 4), in respect of the insertion thereof in said incision. By applying further pressure on the branches 1a, 1b, said branches move closer together resulting in the elastic tightening of the flexible clamping blades 2a, 2b, the grip strength transferred to the ends thereof makes it possible to grip the anterior lens capsule and create the rhexis.

According to one important characteristic feature of the invention, the clamping blades 2a, 2b of the clamp are asymmetrical and oriented in a direction or plane P1 corresponding to the approach and separation plane of the handling branches 1a, 1b, said clamping blades 2a, 2b being arranged on top of each other, such that the approach or separation of said handling branches 1a, 1b results in the approach or separation of the clamping tips 3a, 3b forming the jaws of the clamp, respectively.

According to a further advantageous embodiment, the clamping blade 2a, 2b are designed to be able to slide over each other during the approaching and separation movements of the handling branches 1a, 1b.

According to a further advantageous embodiment, at least one of the clamping blades 2a, 2b is made of an elastically flexible material.

Preferably, both clamping branches 2a, 2b are made of an elastic flexible material.

Advantageously, the microsurgery clamp according to the invention can be manufactured industrially in the form of a single-use, disposable item.

In this case, the handling branches 1a, 1b forming the handle of the clamp may be made of a biocompatible shape memory plastic material, by means of any suitable moulding method whereas the clamping blades 2a, 2b are made of a biocompatible shape memory metal, for example stainless steel.

According to the embodiment illustrated as an example, each of the clamping blades 2a, 2b, comprises a fitted moulded rectilinear proximal portion or securing stem 2c in the distal end of one branch 1a or 1b and a curved distal portion 2d connected to said securing stem by a bend 2e forming an angle therewith, and ending with a clamping tip 3a or 3b, forming one of the jaws of the clamp.

The distal portions 2d of the clamping blades 2a, 2b are tapered and progressively become thinner in the direction of the free end thereof. Said blades have, for example, in this portion, a width varying from 0.6 mm to 0.4 mm, and a thickness of 0.3 mm.

Preferably, the distal portions 2d of the clamping blades 2a, 2b have an identical width, such that, in the clamp closure position, said distal portions are suitable for stacking.

Advantageously, at least one of the clamping blades 2a, 2b has a curved shape.

Preferably, both clamping blades 2a, 2b, have a curved shape, in the portion thereof oriented laterally relative to the handle 1.

In this case, the radius of curvature of the upper clamping blade 2a is smaller than the radius of curvature of the lower clamping blade 2b.

It is observed that the upper clamping blade 2a is shorter than the lower clamping blade 2b.

According to a further characteristic feature, the clamping tip 3b or jaw of the lower clamping blade 2b has, in the upper part thereof, a greater width than that of the distal portion 2d of the upper clamping blade 2a, so as to protrude on either side thereof. The upper portion 3c of the clamping tip 3b of the lower clamping blade 2b has, for example, a width of 0.5 mm. The protruding lateral ends 3c′ of the lower clamping tip 3b can thus be located by the eye surgeon, during the procedure, enabling the surgeon to verify the correct separation (approximately 2 mm) of the clamping tips 3a, 3b required to seize the piece of anterior capsule.

According to a preferred embodiment, the clamping tips 3a, 3b have a triangular shape, and the lower portions thereof are suitable for stacking, in the approach position. The upper clamping tip 3a has, for example, a height of 0.4 mm, whereas the height of the lower clamping tip 3b is 0.2 mm.

The operation of the rhexis clamp according to the invention is described hereinafter. In the idle position, no pressure is applied to the spaced branches 1a, 1b of the clamp, such that the clamping tips 3a, 3b are also spaced.

Approaching the branches 1a and 1b under slight pressure from the surgeon's fingers contacts the clamping blades 2a, 2b and the clamping tips 3a, 3b.

The approach of the branches 1a, 1b, of the clamp results in the application, against each other, of the curved distal portions 2d of the upper 2a and lower 2b distal portions sliding over each other.

Due to the initial difference in curvature of these two portions 2d made of very elastic metal (high spring effect), the lower face of the upper clamping blade 2a will mould perfectly with the shape of the upper face of the lower clamping blade 2b. In this situation, the invasive portion of the rhexis clamp is inserted into the eye via an opening O′ of only 1.5 mm.

During all the following procedures, the width of the invasive portion will remain the same (between 06 and 0.4 mm), enabling the use thereof via this very small incision O′.

In order to be able to grip and release the anterior lens capsule, it should be possible to separate the jaws 3a, 3b of the clamp from each other over a distance in the region of two millimeters.

During the “opening/closing” movement of two mm, no gap remains between the two curved distal portions 2d of the upper 2a and lower 2b clamping blades over a minimum length of 10 mm, from the distal end of the clamping blade 2a thus from the jaw 3a of said upper clamping blade (10 mm “effective” intraocular during the “opening/closing” movement).

The invasive portion 2d-2d of the clip situated in the incision will thus have exactly the same width as the clamp i.e. closed or open by two mm (opening required to grip the capsule).

This invariable width is compatible with a 1.5 mm incision.

If the pressure on the branches 1a, 1b of the clamp is released slightly, separation of the clamping tips 3a, 3b, over a distance in the region of two millimeters is obtained, sufficient to enable the gripping of the anterior lens capsule. This two millimeter separation of the clamping tips 3a, 3b is sufficient to enable the surgeon to easily position same on either side of the capsular piece and seize said piece by approaching said clamping tips 3a, 3b again. In this situation, the clamping force applied by the clamping tips 3a, 3b of the clamping blades 2a, 2b, is sufficient to seize and handle the anterior lens capsule.

1. Microsurgery clamp, particularly a microincision capsulorhexis clamp, comprising a proximal handling portion or handle including two branches or arms interconnected at one of the ends thereof, so as to be suitable for being approached or separated elastically, and a distal clamping portion having two narrow and thin clamping blades connected to the distal ends of the handling branches and oriented laterally relative thereto, the distal end of each of the clamping blades being curved in a direction opposite to that of the handle, so as to form a clamping tip, characterised in that the clamping blades oriented in a direction or plane corresponding to the approaching or separation directions of the handling branches, said clamping blades being arranged above each other, relative to a vertical position of said handle, so that the approaching or separation movement of said handling branches results in the approaching or separation movement of said clamping tips forming the jaws of the clamp, respectively.

2. Microsurgery clamp, particularly a microincision capsulorhexis clamp, according to claim 1, characterised in that the clamping blades can slide over each other during handling branch approaching and separation movements.

3. Microsurgery clamp, particularly a microincision capsulorhexis clamp, according to claim 1, characterised in that at least one of the clamping blades is made of an elastically flexible material.

4. Microsurgery clamp, particularly a microincision capsulorhexis clamp, according to claim 3, characterised in that both clamping blades are made of an elastic flexible material.

5. Microsurgery clamp, particularly a microincision capsulorhexis clamp, according to claim 1, characterised in that at least one of the clamping blades has a curved shape.

6. Microsurgery clamp, particularly a microincision capsulorhexis clamp, according to claim 5, characterised in that both clamping blades have a curved shape.

7. Microsurgery clamp, particularly a microincision capsulorhexis clamp, according to claim 6, characterised in that the radius of curvature of the upper clamping blade is smaller than the radius of curvature of the lower clamping blade.

8. Microsurgery clamp, particularly a microincision capsulorhexis clamp, according to claim 1, characterised in that the upper clamping blade is shorter than the lower clamping blade, in the curved distal portion thereof.

9. Microsurgery clamp, particularly a microincision capsulorhexis clamp, according to claim 1, characterised in that the clamping tip or jaw of the lower clamping blade has, in the upper portion thereof, a greater width than that of the distal portion of the upper clamping blade, so as to protrude on either side thereof.

10. Microsurgery clamp, particularly a microincision capsulorhexis clamp, according to claim 9, characterised in that the clamping tips have a triangular shape and the lower portions thereof are suitable for stacking, in the contact position.
